(19) United States
(12) Patent Application Publication
(10) Pub. No.: US 2012/0020886 A1
(43) Pub. Date: Jan. 26, 2012

(54) THERMAL SPIN CROSS-OVER COMPOUNDS AND METHODS OF USING SAME

(76) Inventor: Chandrasekar RAJADURAI, Andhra Pradesh (IN)

(21) Appl. No.: 12/913,557

(22) Filed: Oct. 27, 2010

(30) Foreign Application Priority Data
Jul. 21, 2010 (IN) 2086/CHE/2010

(51) Int. Cl. A61K 49/00; A61K 31/555; A61P 43/00; C07F 15/02

(52) U.S. Cl. 424/9.1; 546/10; 514/185

(57) ABSTRACT

A compound has the of Formula I or a salt thereof:

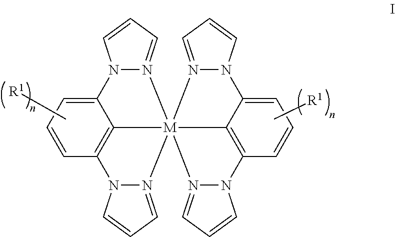

Such compounds may be used to image cells, tissues, organs, fluids, or a subject. Such compounds may also be used in pharmaceutical compositions.

### CROSS-REFERENCE TO RELATED APPLICATIONS

This application claims the benefit of Indian Patent Application No. 2086/CHE/2010, filed Jul. 21, 2010, which is hereby incorporated by reference, in its entirety, for any and all purposes.

### BACKGROUND

Spin cross-over compounds possess molecular bistability which is the ability of a molecular system to be observed in two different electronic states under an external perturbation, such as, but is not limited to, temperature, pressure, and light intensity. Molecular bistability may refer to either a single molecule or to an assembly of molecules.

Spin cross-over compounds are a form of inorganic electronic switches. Variation of a thermal energy at the cross-over may lead to an electronic (change in the d-electron orbital configuration) and structural change, which may be observed as a color and/or a magnetic moment change. Typically, one of the states is the ground state and the other is a metastable state.

The spin-cross-over phenomenon occurs when some molecular species containing an octahedral coordinated transition metal ion with the 3dⁿ (4≦n≦7) electronic configuration may show a cross-over between a low spin (LS) and a high spin (HS) state. The change in the spin state may be associated with a color change.

The spin-cross-over properties of bistable transition metal complexes with high spin cross-over temperature have attracted attention due to their potential application in magnetic resonance imaging (MRI) and visual displays. The spin-cross-over compounds with longer-lived metastable states and larger hysteresis are of interest.

### SUMMARY

In one aspect, a compound represented by Formula I or a salt thereof is provided:

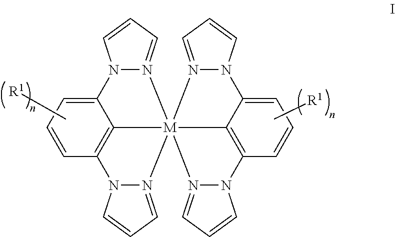

where: n is an integer selected from 1, 2, or 3; M is a transition metal; and each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

In some embodiments, R¹ is halogen, hydroxyl, or carboxyl. In some embodiments, R¹ is halogen.

In some embodiments, M is iron, cobalt, europium, gadolinium, or nickel. In some embodiments, M is iron.

In some embodiments, n is 1.

In some embodiments, a compound represented by Formula I or a salt thereof is provided, where n is 1; M is iron, cobalt, europium, gadolinium, or nickel; and each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

In some embodiments, a compound represented by Formula I or a salt thereof is provided, where n is 1; M is iron; and each R¹ is independently halogen, hydroxyl, or carboxyl.

In some embodiments, a compound represented by Formula I or a salt thereof is provided, where n is 1; M is iron; and each R¹ is a halogen.

In some embodiments, a compound represented by Formula I or a salt thereof is provided, where n is 1; M is iron; and each R¹ is iodo.

In some embodiments, the compound exhibits a temperature dependent reversible switching of the magnetic spin states. In some embodiments, the temperature dependent reversible switching of the magnetic spin states takes place at about room temperature.

In some embodiments, the compound is a contrast agent for magnetic resonance imaging.

In another aspect, a method is provided for preparing the compound represented by Formula I or a salt thereof. In some embodiments, the method includes contacting compound represented by Formula II with a transition metal, M, in a suitable solvent, where Formula II is:

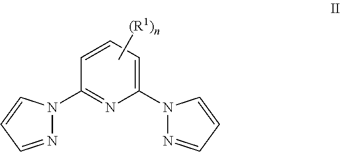

n is 1, 2, or 3; M is a transition metal; and each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

In some embodiments of the method, n is 1; M is iron, cobalt, europium, gadolinium, or nickel; and each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl. In some embodiments, n is 1; M is iron; and R¹ is a halogen. In yet other embodiments, n is 1; M is iron; and R¹ is iodo.

In some embodiments of the method, the suitable solvent is a polar solvent.

In another aspect, a pharmaceutical composition includes any of the compounds of Formula I and a pharmaceutically acceptable carrier; where Formula I is:

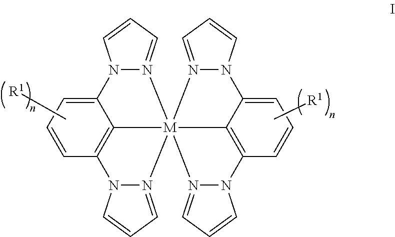

n is 1, 2, or 3; M is a transition metal; and each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

In another aspect, a method is provided for imaging a cell, tissue, organ, fluid, or subject including administering a composition including a compound represented by Formula I, or a salt thereof, to the cell, tissue, organ, fluid, or subject, and imaging the cell, tissue, organ, fluid, or subject. In such embodiments, Formula I is:

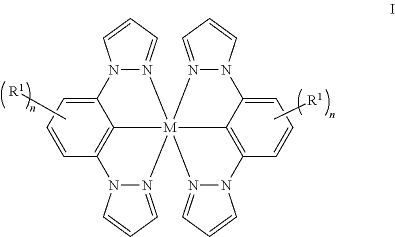

n is 1, 2, or 3; M is a transition metal; and each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

In some embodiments of the method, the method is in vivo, in vitro, or ex vivo.

In some embodiments of the method, the subject has a tumor.

In one aspect of the present technology, a kit is provided for magnetic resonance imaging including a compound represented by Formula I or a salt thereof, optionally a device to dispense the composition; and instructions for use. In such embodiments, Formula I is:

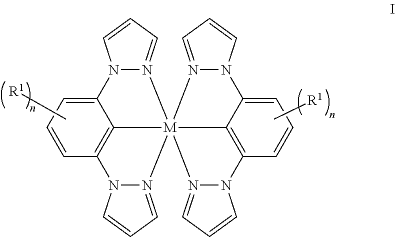

where: n is 1, 2, or 3; M is a transition metal; and each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 depicts an illustrative embodiment of a single crystal X-ray diffraction (XRD) of [Fe^{II}(compound II)₂](BF₄)₂. FIG. 1 is an Oak Ridge Thermal Ellipsoid Plot (ORTEP) of the complex di-cation at 293 K. The di-anions (two BF₄ negative counter ions) and the hydrogens are omitted for clarity. Bond distances: Fe—N(1): 1.974 Å; Fe—N(2): 1.909 Å; Fe—N(3): 1.976 Å.

FIG. 2A and FIG. 2B depict an illustrative embodiment of a χT=f(T) plot of powder sample of [Fe^{II}(compound II)₂](BF₄)₂.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present technology is described herein using several definitions, as set forth throughout the specification. As used herein, unless otherwise stated, the singular forms “a,” “an,” and “the” include plural reference.

The term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this technology or process steps to produce a composition or achieve an intended result. Embodiments defined by each of these transition terms are within the scope of this technology.

One aspect of the present technology includes thermal spin cross-over (SCO) compounds which exhibit temperature dependent reversible switching of the magnetic spin states (high spin, paramagnetic [symbol] low spin, diamagnetic). The SCO compounds may change their spin states depending on the temperature, from the low spin (LS) state at low temperature to high spin (HS) state at high temperature. In some embodiments, the SCO compounds of the present technology exhibit the switching temperature (T_{1/2}) at above room temperature. In some embodiments, the spin cross-over is associated with a color change which makes the compounds suitable candidates for use as a contrast agent in magnetic resonance imaging (MRI).

For example, the compound of Formula III (Example 1 below) demonstrates spin transition temperature (T_{1/2}) of 328 K or 45° C. The compound is paramagnetic above human body temperature (ca. 310 K or 27° C.) and diamagnetic at and below human body temperature. Therefore, the compound of Formula III can be used to image (MRI) cancer cells or to measure intercellular temperature of cells within human body with MRI technique since only the paramagnetic state may give the magnetic contrasts and not the diamagnetic one.

The compounds of the present technology exhibit unexpected properties including, but are not limited to, spin transition at above room temperature, a thermally induced spin-state transition occurring over a narrower temperature range, and a wide hysteresis loop. The unexpected properties of the compounds of the present technology facilitate their use as a contrast agent.

In some embodiments, the SCO compounds of the present technology find application in MRI as contrast agents. By way of example only, the magnetic spin state of the SCO compound may switch automatically depending on the target cell temperature, e.g. tumor cells of animal or human, where the temperature is typically higher. Such switching of the spin states may be associated with a color change, thereby making the compounds effective contrast agents.

In MRI, a proton relaxation rate may be measured for the contrast agents. The SCO compounds show temperature dependent relaxation rates at different temperatures due to the change in spin states. The relaxation rate may be high at high spin state at high temperature and low at low spin state at low temperature. When the SCO compound reaches a target tumor cell of the animal or human origin, the compound may switch from low spin state to high state. This may enhance the proton relaxation rate and a MRI contrast image may be obtained as compared to normal cells.

The spin state of the spin-cross-over compounds involving the Fe²⁺ ion with the 3d⁶ configuration is characterized by complete spin pairing in the low spin state, and with one pair of electrons and four unpaired electrons in the high spin state. The spin state on the Fe^{II} ion changes from diamagnetic (S=0) in the LS state, to paramagnetic (S=2) in the HS state. The Fe^{II} spin transition may be accompanied by a color change. This cross-over may be induced by variation of temperature, pressure, or radiation intensity.

In octahedral surroundings, the 3d metal orbitals are split into the low-lying t_{2g} and high-lying e_{g} subsets. The LS state arises from the close-shell t_{2g}⁶ electronic configuration and the HS state from the t_{2g}⁴e_{g}² electronic configuration. In the HS state, the antibonding e_{g} orbitals are doubly occupied, which results in a lengthening of the Fe-ligand bonds, compared with the LS state, typically by 0.15 to 0.18 Å.

In some embodiments, the compounds of the present technology exhibit high water solubility thereby facilitating ease of use in in vitro systems or ease of delivery in in vivo systems. The high water solubility of the compounds of the present technology also results in ease of distribution of the compound in the body of the subject.

Provided herein are spin cross-over compounds and their compositions, method of preparation, method of use, and kits.

In one aspect of the present technology, there is provided a compound of Formula I or a salt thereof:

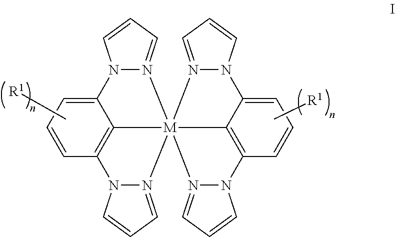

where: n is 1, 2, or 3; M is a transition metal; and each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

The “salt” includes pharmaceutically acceptable salts of the compound of formula I. The salt of the compound of formula I includes the corresponding anion of the transition metal. For example, the salt includes the tetrafluoroborate anion of the compound of formula I when M is Fe. The salts may also be derived from a variety of organic and inorganic counter ions well known in the art and include, by way of example only, sodium, potassium, calcium, magnesium, ammonium, and tetraalkylammonium. When the compound contains a basic functionality then salts of organic or inorganic acids include, such as, but are not limited to, hydrochloride, hydrobromide, tartrate, mesylate, acetate, maleate, and oxalate (see Stahl and Wermuth, eds., “Handbook Of Pharmaceutically Acceptable Salts,” (2002), Verlag Helvetica Chimica Acta, Zurich, Switzerland, for an extensive discussion of pharmaceutical salts, their selection, preparation, and use).

Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydroiodic acid, etc.), sulfuric acid, nitric acid, phosphoric acid, and the like.

The term “transition metal” refers to “an element whose atom has an incomplete d sub-shell, or which can give rise to cations with an incomplete d sub-shell.”

The term “halogen” or “halo” refers to fluoro, chloro, bromo, and iodo.

The term “hydroxyl” or “hydroxy” refers to group —OH.

The term “C₁₋₆ alkyl” refers to monovalent saturated aliphatic hydrocarbyl groups having from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), neopentyl ((CH₃)₃CCH₂—), and hexyl.

The term “C₁₋₆ substituted alkyl” refers to an alkyl group having from 1 to 5, alternatively 1 to 3, or alternatively 1 to 2 substituents selected from the group consisting of halogen, hydroxyl, alkyl, alkoxy, substituted alkoxy, carboxyl, amine, substituted amine, amide, substituted amide, thiol, and thioalkyl.

Alkoxy includes, by way of example, methoxy, ethoxy, n-propoxy, isopropoxy, n-butoxy, t-butoxy, sec-butoxy, and n-pentoxy.

The term “C₁₋₆ substituted alkoxy” refers to the group —O—(C₁₋₆ substituted alkyl) wherein C₁₋₆ substituted alkyl is defined herein.

The term “acyl” refers to the groups H—C(O)—, alkyl-C(O)—, substituted alkyl-C(O)—, alkoxy-C(O)—, or substituted alkoxy-C(O)—. Acyl includes the acetyl group CH₃C(O)—.

The term “carboxyl” or “carboxy” refers to the group —COOH or salts thereof or —COO-alkyl, where alkyl is as defined herein.

The term “amine” or “amino” refers to the group —NH₂.

The term “substituted amine” or “substituted amino” refers to the group —NR¹⁰R¹¹ where R¹⁰ and R¹¹ are independently selected from the group consisting of hydrogen, alkyl, substituted alkyl, alkoxy, substituted alkoxy, halogen, hydroxyl, or acyl.

The term “amide” refers to the group —CONH₂.

The term “substituted amide” refers to the group —CONR¹²R¹³ where R¹² and R¹³ are independently selected from the group consisting of hydrogen, alkyl, substituted alkyl, alkoxy, substituted alkoxy, halogen, hydroxyl, or acyl.

The term “thiol” refers to the group —SH.

The term “thioalkyl” refers to the group —S-alkyl, where alkyl is as defined herein.

It is understood that the above definitions are not intended to include impermissible substitution patterns (e.g., methyl substituted with 5 fluoro groups). Such impermissible substitution patterns are well known to the skilled artisan.

In some embodiments, R¹ is halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

In some embodiments, R¹ is halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, or substituted amide.

In some embodiments, R¹ is halogen, hydroxyl, C₁₋₆ alkyl, C₁₋₆ substituted alkyl, C₁₋₆ alkoxy, C₁₋₆ substituted alkoxy, acyl, or carboxyl.

In some embodiments, R¹ is halogen, hydroxyl, C₁₋₆ alkyl, C₁₋₆ substituted alkyl, C₁₋₆ alkoxy, or C₁₋₆ substituted alkoxy.

In some embodiments, R¹ is halogen, hydroxyl, or carboxyl. In some embodiments, R¹ is hydroxyl, or carboxyl.

In some embodiments, R¹ is halogen. In some embodiments, halogen is selected from the group consisting of fluoro, bromo, chloro, or iodo. In some embodiments, halogen is bromo or iodo. In some embodiments, halogen is iodo.

In some embodiments, M is a transition metal. Such transition metals may include iron, cobalt, europium, gadolinium, or nickel. In some embodiments, M is iron. In some embodiments, M is iron, cobalt or copper. In some embodiments, M is iron, europium, or gadolinium. In some embodiments, M is iron or cobalt. In some embodiments, M is nickel.

In some embodiments, n is 1. In some embodiments, n is 2. In some embodiments, n is 3.

In some embodiments, there is provided a compound of Formula I or a salt thereof, wherein n is 1; M is iron, cobalt, europium, gadolinium, or nickel; and each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

In some embodiments, there is provided a compound of Formula I or a salt thereof, wherein n is 1; M is iron; and each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

In some embodiments, there is provided a compound of formula I or a salt thereof, wherein n is 1; M is iron; and each R¹ is independently halogen, hydroxyl, or carboxyl.

In some embodiments, there is provided a compound of formula I or a salt thereof, wherein n is 1; M is iron, cobalt, europium, gadolinium, or nickel; and each R¹ is independently halogen, hydroxyl, or carboxyl.

In some embodiments, there is provided a compound of formula I or a salt thereof, wherein n is 1; M is iron; and each R¹ is a halogen.

In some embodiments, there is provided a compound of formula I or a salt thereof, wherein n is 1; M is iron; and each R¹ is iodo.

In some embodiments, the compound exhibits a temperature dependent reversible switching of the magnetic spin states. In some embodiments, the compound exhibits a temperature dependent reversible switching of the magnetic spin states from low spin state at lower temperature to high spin state at higher temperature. In some embodiments, the compound exhibits a temperature dependent reversible switching of the magnetic spin states between the temperature of about 322K-334K, at ambient pressure. The temperature range may increase or decrease depending upon the external pressure applied and the number of times the magnetic measurement is performed. In some embodiments, the compound exhibits a temperature dependent reversible switching of the magnetic spin states between about 322K-324K; or about 322K-328K; or about 324K-328K; or about 324K-334K; or about 328K-334K; or about 325K-330K; or about 324K; or about 328K; or about 330K; or about 332K; or about 334K. In some embodiments, the compound exhibits the temperature dependent reversible switching of the magnetic spin states at about room temperature. In some embodiments, the compound exhibits the temperature dependent reversible switching of the magnetic spin states at above room temperature.

In some embodiments, the compound exhibits a wide thermal hysteresis loop. In some embodiments, the compound exhibits a wide thermal hysteresis loop of between about 8K-12K, at ambient pressure. Again, this range may vary depending upon the external pressure applied during measurement. In some embodiments, the compound exhibits a wide thermal hysteresis loop of between about 8K-12K; or between about 8K-10K; or between about 10K-12K; or about 10K; or about 11K or about 12K.

In some embodiments, the compound is a contrast agent for magnetic resonance imaging.

In one aspect of the present technology, there is provided a method of preparing a compound of Formula I or a salt thereof. Such embodiments include contacting compound represented by Formula II with a transition metal, M, in a suitable solvent. Formula II is:

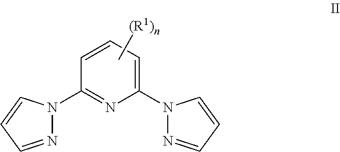

where n is 1, 2, or 3; and each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl. Any of the compounds represented by Formula I as described above can be prepared using the methods described herein.

In some embodiments of the methods aspect of the present technology, the suitable solvent is a polar solvent. Examples of the polar solvent include, but are not limited to, acetonitrile, dichloromethane, alcohol, dimethylformamide, dimethylsulfoxide, and ethyl acetate.

In some embodiments, the method of preparing a compound of Formula I or a salt thereof includes heating a reaction mixture to reflux or stirring the reaction mixture at room temperature.

In some embodiments, the method of preparing a compound of Formula I or a salt thereof further includes one or more of standard synthetic techniques, such as, but not limited to, filtration, evaporation of the solvent, purification, crystallization, etc. The compound can be characterized based on standard spectroscopic data generated from spectroscopic techniques, including, but not limited to, mass spectroscopy, X-ray diffraction (XRD), nuclear magnetic resonance (NMR), etc.

The thermal spin cross-over (SCO) transition metal compounds of the present technology can be used as contrast agents for MRI. The compounds exhibiting temperature dependent reversible switching of the magnetic spin states (high spin, paramagnetic [symbol] low spin, diamagnetic), may result in color change at about or above room temperature thereby facilitating their use as a contrast agent in MRI. The compounds can be used in detecting tumor in the cell, tissue, organ, fluid, or subject to help a physician in diagnosing and treating cancer related conditions.

Various conditions that can be diagnosed and treated using the methods described herein include, but are not limited to, tumors of the chest, abdomen or pelvis; certain types of heart problems; blockages or enlargements of blood vessels, including the aorta, renal arteries, and arteries in the legs; diseases of the liver, such as cirrhosis, and that of other abdominal organs, including the bile ducts, gallbladder, and pancreatic ducts; diseases of the small intestine, colon, and rectum; cysts and solid tumors in the kidneys and other parts of the urinary tract; tumors and other abnormalities of the reproductive organs (e.g., uterus, ovaries, testicles, prostate); causes of pelvic pain in women, such as fibroids, endometriosis and adenomyosis; suspected uterine congenital abnormalities in women undergoing evaluation for infertility; and breast cancer and implants.

The “subject” of diagnosis or treatment refers to an animal such as a mammal, or a human. Non-human animals subject to diagnosis or treatment include, for example, simians, murine, such as, rats, mice, canine, such as dogs, leporids, such as rabbits, livestock, sport animals, and pets. The “cell,” “tissue,” “organ,” or a “fluid” may independently correspond to any of the above noted subject. The “fluid” may be selected from blood, urine, sweat, saliva, etc.

The compound(s) described herein, or compositions thereof, will generally be used in an amount effective to achieve the intended result, for example, in an amount effective for use as a contrast agent in MRI. The compound(s) can be administered in an diagnostically effective amount. A “diagnostically effective amount” refers to the amount of a compound or the composition of the present technology to facilitate a desired diagnostic result. Diagnostics includes testing that is related to the in vitro, ex vivo, or in vivo diagnosis of disease states or biological status (e.g. tumor) in mammals, for example, but not limited to, humans. The effective amount will vary depending upon the specific compound or composition used, the dosing regimen, timing of administration, the subject and disease condition being diagnosed, the weight and age of the subject, the severity of the disease condition, the manner of administration and the like, all of which can be determined readily by one of ordinary skill in the art.

The amount of compound administered may depend upon a variety of factors, including, for example, the particular condition being diagnosed, the mode of administration, the severity of the condition being diagnosed, the age and weight of the patient, the bioavailability of the particular compound. Determination of an effective dosage is well within the capabilities of those skilled in the art. As known by those of skill in the art, the preferred dosage of compounds of the present technology will also depend on the age, weight, general health, and severity of the condition of the individual being diagnosed. Dosage may also need to be tailored to the sex of the individual. Dosage, and frequency of administration of the compounds or salts thereof, may also depend on whether the compounds are formulated for diagnosis of acute episodes of a condition. A skilled practitioner will be able to determine the optimal dose for a particular individual.

The efficiency of the compounds may be investigated by involving a concept of relaxivity, referring to the nuclear relaxation enhancement normalized to 1 mM concentration of the magnetic species. At not too high concentration of the paramagnetic species, the enhancement is proportional to that concentration. The magnetic species may enhance the proton relaxation rates due to a random variation of the electron spin-nuclear spin interactions (the dipole-dipole interaction and the magnetic hyperfine interaction between the nuclear and electron magnetic moments), which may open pathways for longitudinal as well as transverse relaxation. Measurements of the relaxation enhancement or relaxivity over a broad range of magnetic fields are referred to as relaxometry, and the resulting curve is denoted as a nuclear magnetic relaxation dispersion (NMRD) profile.

In one aspect of the present technology, there is provided a method for imaging a cell, tissue, organ, fluid, or subject. Where a cell, tissue, organ, or fluid is to be imaged, the compound represented by Formula I, or a salt thereof, is contacted with the cell, tissue, organ, or fluid, and the imaging is conducted. Where a subject is to be imaged, the compound represented by Formula I, or a salt thereof, is administered to the subject and the subject is imaged. In some embodiments, the method is conducted in vivo, in vitro, or ex vivo. In some embodiments, the subject has a tumor and the tumor is imaged in vivo.

In one aspect of the present technology, there is provided a pharmaceutical composition including any of the compounds represented by Formula I or a salt thereof as described above, and a pharmaceutically acceptable carrier.

A “carrier” refers to any diluents, excipients, or carriers that may be used in the compositions of the present technology. A “pharmaceutically acceptable carrier” refers to a carrier that is acceptable for any pharmaceutical use. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, and polyethylene glycol. Suitable pharmaceutical carriers are described in Remington's Pharmaceutical Sciences, Mack Publishing Company, a standard reference text in this field. They are preferably selected with respect to the intended form of administration, that is, oral elixirs, syrups, injectable vehicle and the like, and consistent with conventional pharmaceutical practices.

Pharmaceutical compositions including the compounds described herein (or salts thereof) can be manufactured by means of conventional mixing, dissolving, granulating, dragee-making, levigating, emulsifying, encapsulating, entrapping, or lyophilization processes. The compositions can be formulated in conventional manner using one or more physiologically acceptable carriers, diluents, excipients, or auxiliaries which facilitate processing of the active compounds into preparations which can be used pharmaceutically.

The compounds described herein can be administered by oral or parenteral routes (e.g., intramuscular, intraperitoneal, intravenous, intracisternal injection or infusion, subcutaneous injection, or implant), and can be formulated, alone or together, in suitable dosage unit formulations containing conventional non-toxic pharmaceutically acceptable carriers, adjuvants, excipients, and vehicles appropriate for each route of administration.

The pharmaceutical compositions for the administration of the compounds can be conveniently presented in dosage unit form and can be prepared by any of the methods well known in the art of pharmacy. The pharmaceutical compositions can be, for example, prepared by uniformly and intimately bringing the active ingredient into association with a liquid carrier, a finely divided solid carrier or both, and then, if necessary, shaping the product into the desired formulation. In the pharmaceutical composition, the active object compound is included in an amount sufficient to produce the desired therapeutic effect. For example, pharmaceutical compositions may take a form suitable for virtually any mode of administration, including, for example, oral, systemic, injection, or infusion.

Systemic formulations include those designed for administration by injection (e.g., subcutaneous, intravenous, intramuscular, intrathecal, or intraperitoneal injection) as well as those designed for oral administration.

Useful injectable preparations include sterile suspensions, solutions, or emulsions of the compound(s) in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powdered form for reconstitution with a suitable vehicle, including but not limited to sterile pyrogen free water, buffer, and dextrose solution, before use. To this end, the compound(s) can be dried by any art-known technique, such as lyophilization, and reconstituted prior to use. The formulation can also be provided as a tablet or capsule.

The oral formulations include liquids or syrups. Compositions intended for oral use can be prepared according to any method known in the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. The pharmaceutical compositions described herein may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate. Preparations for oral administration can be suitably formulated to give controlled release or sustained release of the active compound, as is well known.

The pharmaceutical compositions can be in the form of a sterile injectable aqueous or oleaginous suspension. This suspension can be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that can be employed are water, Ringer's solution, and isotonic sodium chloride solution.

As those skilled in the art will recognize, the formulation of compounds, the quantity of the formulation delivered, and the duration of the administration of a single dose depend on various factors, including, but are not limited to, type of a cell, tissue, organ or subject as well the solubility of the compound and/or the transition metal in the compound. In some embodiments, the frequency of administration and length of time for which the compound is administered will depend on the concentration of compounds in the formulation. For example, shorter periods of administration can be used at higher concentrations of compounds.

For prolonged delivery, the compound(s) described herein can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives (e.g., as a sparingly soluble salt).

Alternatively, other pharmaceutical delivery systems can be employed. Liposomes and emulsions are well-known examples of delivery vehicles that can be used to deliver active compound(s).

The pharmaceutical compositions may, if desired, be presented in a pack or dispenser device which may contain one or more unit dosage forms containing the compound(s). The pack may, for example, include metal or plastic foil, such as a blister pack. The pack or dispenser device can be accompanied by instructions for administration.

Effective dosages can be estimated initially from in vitro assays. For example, an initial dosage for use in animals can be formulated to achieve a circulating blood or serum concentration of active compound as measured in in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” Goodman And Gilman's The Pharmaceutical Basis Of Therapeutics, Chapter 1, pp. 1-46, latest edition, Pergammon Press, and the references cited therein.

Initial dosages can also be estimated from in vivo data, such as animal models. Animal models useful for testing the efficacy of compounds as contrast agents are well-known in the art. Ordinarily skilled artisans can routinely adapt such information to determine dosages suitable for human administration.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) which are sufficient to maintain diagnostic effect. For example, the compounds can be administered once per week, several times per week (e.g., every other day), once per day, or multiple times per day, depending upon, among other things, the mode of administration, the specific indication being diagnosed, and the judgment of the prescribing physician. In cases of local administration or selective uptake, such as local injection, the effective local concentration of the compound(s) may not be related to plasma concentration. Skilled artisans will be able to optimize effective local dosages without undue experimentation.

The compound(s) may provide diagnostic benefit without causing substantial toxicity. Toxicity of the compound(s) can be determined using standard pharmaceutical procedures. The dose ratio between toxic and diagnostic effect is the index. Compounds(s) that exhibit high indices are preferred.

The foregoing disclosure pertaining to the dosage requirements for the compounds of the present technology is pertinent to dosages required for salts of the compounds, with the realization, apparent to the skilled artisan, that the amount of salt administered will also depend upon a variety of factors, including, for example, the bioavailability of the particular salt and the conversation rate and efficiency into the compound under the selected route of administration. Determination of an effective dosage of salt for a particular use and mode of administration is well within the capabilities of those skilled in the art.

In one aspect of the present technology, there is provided a kit for magnetic resonance imaging including a compound represented by Formula I or a salt thereof as described above, optionally a device to dispense the compound; and instructions for use.

The kits may further include suitable packaging and/or instructions for use of the compound or composition. The device to dispense the compound or the composition includes, but is not limited to, syringe, catheter, or other such devices. The kits may further include surgical tools.

The kits may also include other agents for use in conjunction with the compound or composition described herein. Such agents include, but are not limited to, e.g., alcohol, analgesics, anesthetics, antiseptics, etc. These agents can be provided in a separate form. The kits may include appropriate instructions for the use of the compound or the composition, side effects, and any other relevant information. The instructions can be in any suitable format, including, but not limited to, e.g., printed matter, videotape, or computer readable disk.

In some embodiments, the compound or composition or the other agents may be present in a vial, pouch, leaf, ampoule, container, syringe, or any other means for carrying the compound or the composition.

Other types of kits provide the compound and reagents to prepare a composition for administration. The composition can be in a dry or lyophilized form or in a solution, particularly a sterile solution. When the composition is in a dry form, the reagent may include a pharmaceutically acceptable diluent for preparing a liquid formulation. The kit may contain a device for administration or for dispensing the compositions, including, but not limited to, syringe, catheter, and/or pipette.

In another embodiment, there is provided a kit including the formulation including a compound selected from the compounds described herein or a salt thereof and at least one pharmaceutically acceptable excipient, diluent, preservative, stabilizer, or mixture thereof, packaging, and instructions for use. In another embodiment, kits for treating an individual who suffers from the conditions described herein are provided, including a container including a dosage amount of a compound or composition, as disclosed herein, and instructions for use. The container can be any of those known in the art and appropriate for storage and delivery of oral or injectable formulations.

The compounds described herein can be synthesized via a variety of different synthetic routes using commercially available starting materials and/or starting materials prepared by conventional synthetic methods. It will also be appreciated by those skilled in the art that in the process described below, the functional groups of intermediate compounds may need to be protected by suitable protecting groups.

The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, Protective Groups In Organic Synthesis, 3d Edition, John Wiley & Sons, Inc., New York (1999) and the references cited therein. Examples of functional groups include hydroxy, amino, thio, and carboxylic acid.

Thus, “protecting group” refers to a group of atoms that, when attached to a reactive functional group in a molecule, mask, reduce or prevent the reactivity of the functional group. Typically, a protecting group can be selectively removed as desired during the course of a synthesis. Examples of protecting groups can be found in Greene and Wuts, as mentioned above, and, additionally, in Harrison et al., Compendium Of Synthetic Organic Methods, Vols. 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”), and the like. Representative hydroxyl protecting groups include, but are not limited to, those where the hydroxyl group is either acylated to form acetate and benzoate esters or alkylated to form benzyl and trityl ethers, as well as alkyl ethers, tetrahydropyranyl ethers, trialkylsilyl ethers (e.g., TMS or TIPPS groups), aryl silyl ethers (e.g., triphenylsilyl ether), mixed alkyl and aryl substituted silyl ethers, and allyl ethers.

The following reaction Scheme illustrates methods to make compounds described herein. It is understood that one of ordinary skill in the art would be able to make the compounds by similar methods or by methods known to one skilled in the art. In general, starting components may be obtained from sources such as Aldrich, or synthesized according to sources known to those of ordinary skill in the art (see, e.g., Smith and March, March's Advanced Organic Chemistry: Reactions, Mechanisms, And Structure, 5th edition (Wiley Interscience, New York)). Moreover, the substituted groups (e.g., R¹) of the compounds of the present technology may be attached to the starting components, intermediate components, and/or final products according to methods known to those of ordinary skill in the art.

In one exemplary embodiment, compounds represented by Formula I can be synthesized according to Scheme I.

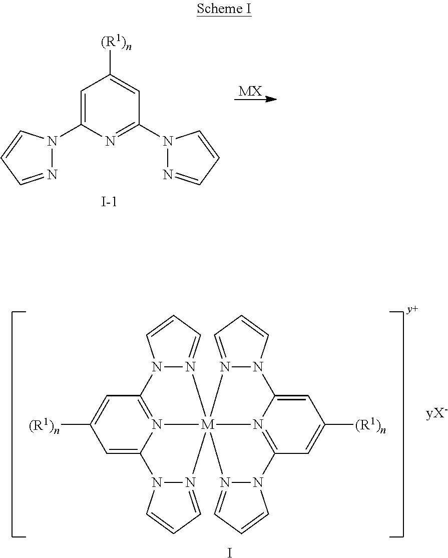

In Scheme I, the groups n, M and R¹ are as defined herein. X is the corresponding anion of the transition metal. For example, MX can be Fe(BF₄)₂ where M is Fe²⁺ and X is BF₄⁻. It is to be understood that the anions X attached to the transition metal M may vary and may be selected from common anions known in the art. The integer y in the compound of formula I may vary depending on the oxidation state of the transition metal ion. The y number of anions for X will vary according to the charge of M.

The starting compounds I-1 can be purchased from commercial sources or prepared using standard techniques of organic chemistry. For example, compound of formula I-1 when R¹ is iodo group, can be synthesized as per the procedure described in Rajadurai et. al. Inorg. Chem. 2006, 45, 10019.

The iodo group in the compound of formula I-1 or compound of formula III (described below) may be substituted with a nucleophile using standard nucleophilic substitution reactions. Such substitution reactions are well known in the art. See also Vogel, 1989, Practical Organic Chemistry, Addison Wesley Longman, Ltd. and John Wiley & Sons, Inc. Such reactions can be used to substitute iodo with hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, and thioalkyl.

The compound I-1 is then reacted with the transition metal MX in the presence of a suitable solvent and under suitable reaction conditions. The suitable solvent includes any polar solvent, such as, but is not limited to, acetonitrile, dimethylformamide, etc. The suitable reaction conditions include, but are not limited to, stirring at room temperature or at high temperature, such as, reflux for 0-48 hrs depending on the starting materials and the solvent.

In each of the above recited steps, the product may be recovered by conventional methods such as evaporation, chromatography, precipitation, crystallization, and the like or, alternatively, used in the next step without purification and/or isolation. The reactions depicted in Scheme I may proceed more quickly when the reaction solutions are rapidly heated by, e.g., a microwave.

The following examples are intended to illustrate the various embodiments of the present technology.

### EXAMPLES

The present technology is further illustrated by the following examples, which should not be construed as limiting in any way. In the examples below as well as throughout the application, the following abbreviations have the following meanings. If not defined, the terms have their generally accepted meanings.

### Example 1

### Synthesis of [Fe^{II}(compound II)₂](BF₄)₂

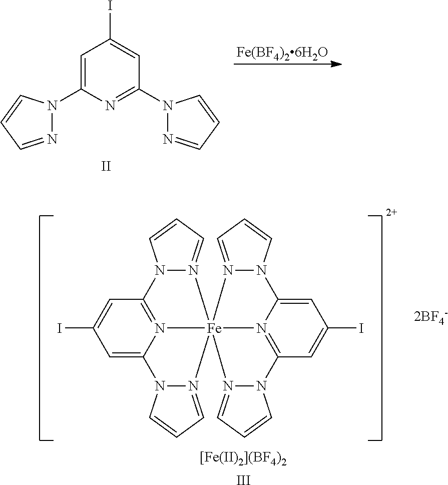

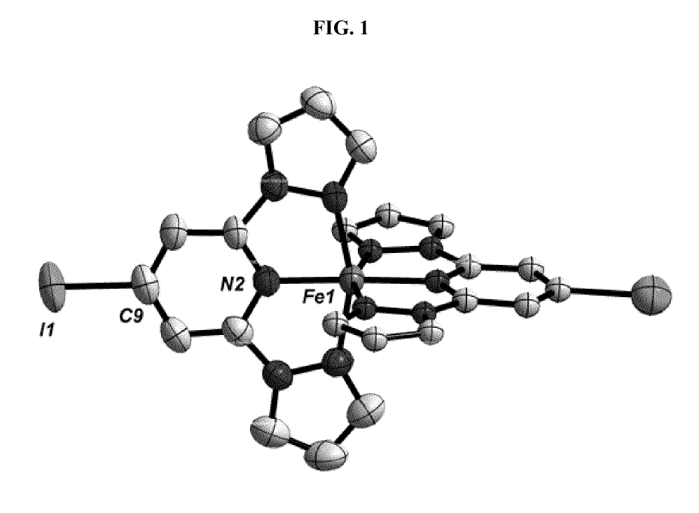

The ligand 4-iodo-2,6-bis(pyrazol-1-yl)pyridine II (R¹═I) was synthesized as per the procedure described in Rajadurai et. al. Inorg. Chem. 2006, 45, 10019. A 84.5 mg (0.25 mmol) of 2,6-bis-pyrazolyl-4-iodopyridine was dissolved in 30 mL of deaerated acetonitrile. Fe(BF₄)₂.6H₂O (41.93 mg, 0.125 mmol) was added to the above solution. The color of the solution immediately turned orange red. The solution was kept under reflux accompanied with stirring for overnight under nitrogen atmosphere. After completion of the reaction, the orange red color solution was filtered and to the filtrate 250 mL di-isopropylether was added to precipitate out the orange red color complex. The precipitated complex was washed with di-isopropylether (3×30 mL) to get a red color powder in 68% yield. (76.7 mg). The complex powder was re-dissolved in acetonitrile and kept for crystallization by slowly diffusing diisopropylether vapor into it. Bright red colored crystals were formed after seven days. A single crystal X-ray diffraction structure obtained at room temperature is illustrated in FIG. 1.

At 293 K, the single crystal XRD structure revealed an octahedral coordination environment of the complex (FIG. 1). The Fe—N distances varied from 1.909 to 1.976 Å, indicating the presence of an iron(II) ion in the low-spin (LS) state at 293 K. This also demonstrated that the metastable high-spin (HS) state is above room temperature indicating that the SCO or spin transition temperature is about or slightly above room temperature.

### Example 2

### Synthesis of Other Compounds

The compounds of formula I, where R¹ is other than iodo, can be prepared from the compound of formula II using standard nucleophilic substitution reactions where the iodo substituent in the compound of formula II is replaced by a desired nucleophile. Such reactions are well known to the skilled artisan. Alternatively, compounds of formula I can be prepared by carrying out the reaction of Example 1 on starting compounds of formula II except that the iodo substituent is replaced with the desired group such as hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, and thioalkyl.

### Example 3

### Magnetic Measurement Study

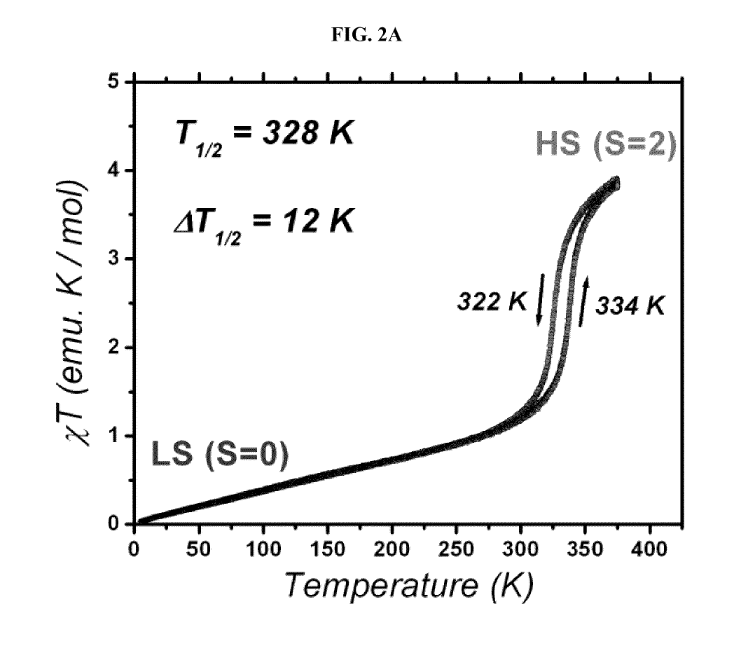

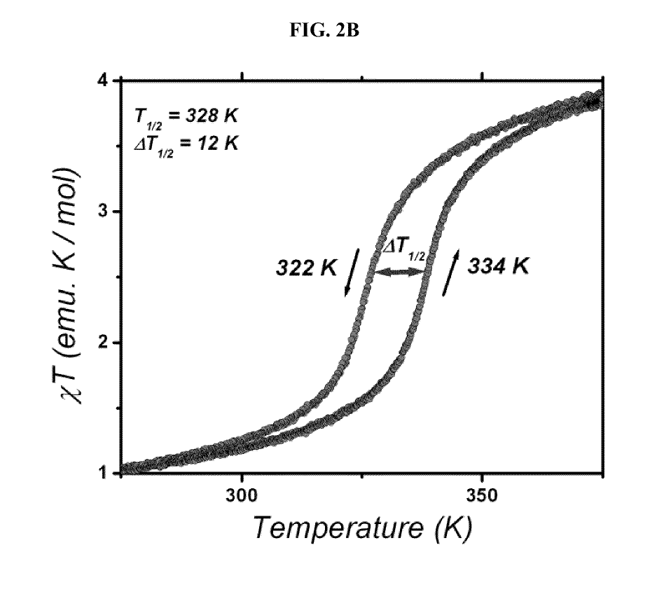

The variable temperature magnetic measurement of [Fe^{II}(compound II)₂](BF₄)₂ was performed on a VSM-SQUID (Vibrating Sample Magnetometer-Super Conducting Quantum Interference Device) set-up in a temperature range of 5 [symbol] 375 K with an applied DC magnetic field of 100 oersted (Oe) (FIGS. 2A and 2B).

At 375 K, the product of molar magnetic susceptibility and temperature, χT, was 3.88 emu·K/mol, which was close to the expected value for a high-spin (HS; S=2) state iron(II) ion. Upon cooling, χT abruptly decreased to a value of 1.22 emu·K/mol at 300 K and reached a minimum value of ca. 0.01 emu·K/mol with a slow and steady decrease at 5 K. The latter minimum value can be attributed to the iron(II) LS (S=0) state. Measurements performed in both cooling (↓) and heating (↑) cycles demonstrated the occurrence of a ca. 12 K (ΔT_{1/2}) wide thermal hysteresis loop (T_{1/2}↓=322 K or 39° C. and T_{1/2}↑=334 K or 51° C.). Notably, the spin transition (ST) temperature was above room temperature with a T_{1/2} of 328 K. The presence of a thermal hysteresis loop clearly demonstrated the existence of a significant level of intermolecular cooperativity in the solid state.

### EQUIVALENTS

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A compound of Formula I or a salt thereof: 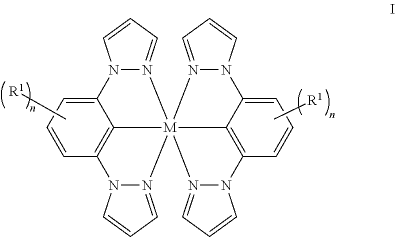
wherein
n is 1, 2, or 3;
M comprises a transition metal; and
each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

2. The compound of claim 1, wherein R¹ is halogen, hydroxyl, or carboxyl.

3. The compound of claim 1, wherein R¹ is halogen.

4. The compound of claim 1, wherein M is iron, cobalt, europium, gadolinium, or nickel.

5. The compound of claim 1, wherein M is iron.

6. The compound of claim 1, wherein n is 1.

7. The compound of claim 1 or the salt thereof, wherein
n is 1;
M is iron, cobalt, europium, gadolinium, or nickel; and
each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

8. The compound of claim 1 or the salt thereof, wherein
n is 1;
M is iron; and
each R¹ is independently halogen, hydroxyl, and carboxyl.

9. The compound of claim 1 or the salt thereof, wherein
n is 1;
M is iron; and
each R¹ is a halogen.

10. The compound of claim 1 or the salt thereof, wherein
n is 1;
M is iron; and
each R¹ is iodo.

11. The compound of claim 1, wherein the compound exhibits temperature dependent reversible switching of the magnetic spin states.

12. The compound of claim 11, wherein said temperature dependent reversible switching of the magnetic spin states takes place at about room temperature.

13. The compound of claim 1, wherein the compound is a contrast agent for magnetic resonance imaging.

14. A method of preparing the compound of claim 1, comprising:
contacting a compound of Formula II with a transition metal, M, in a suitable solvent;
wherein:
Formula II is: 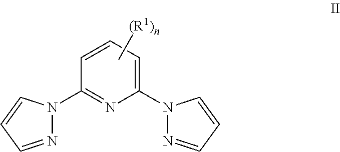
n is 1, 2, or 3; and
each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

15. The method of claim 14, wherein;
n is 1;
M is iron, cobalt, europium, gadolinium, or nickel; and
Each R¹ is independently halogen, hydroxyl, C₁₋₆ alkyl, C₁₋₆ substituted alkyl, C₁₋₆ alkoxy, C₁₋₆ substituted alkoxy, acyl, carboxyl, amine, substituted amine, amide, substituted amide, thiol, or thioalkyl.

16. The method of claim 14, wherein
n is 1;
M is iron; and
R¹ is a halogen.

17. The method of claim 14, wherein
n is 1;
M is iron; and
R¹ is iodo.

18. The method of claim 14, wherein the suitable solvent is a polar solvent.

19. A pharmaceutical composition, comprising the compound of claim 1 and a pharmaceutically acceptable carrier.

20. A method for imaging a subject, comprising administering the compound of claim 1 to the subject and imaging the subject.
